(19) United States
(12) Patent Application Publication
(10) Pub. No.: US 2001/0006949 A1
(43) Pub. Date: Jul. 5, 2001

(54) METHODS AND COMPOSITIONS FOR PRODUCING CELLS RESISTANT TO APOPTOSIS

(76) Inventors: BERNARD THORENS, EPALINGES (CH); PHILLIPPE DUPRAZ, CRISSIER (CH); WILLIAM-FRANCOIS PRALONG, GENEVE (CH)

Correspondence Address: IVOR R ELRIFI, MINTZ LEVIN COHN FERRIS GLOVSKY AND, POPEO PC, ONE FINANCIAL CENTER, BOSTON, MA, 02111

(21) Appl. No.: 09/219,189

(22) Filed: Dec. 23, 1998

(51) Int. Cl. A61K 48/00

(52) U.S. Cl. 514/44; 536/23.5; 435/325; 435/395; 435/397; 435/398

(57) ABSTRACT

The present invention provides genetically engineered cell lines that expresses bcl-2 in response to hypoxic conditions and are, therefore, resistant to apoptosis. In one embodiment, βTc-tet cells are stably transformed with the bcl-2 gene operably linked to the hypoxia responsive PGK promoter. The cells may be provided directly to a patient or may be encapsulated to from a bioartificial organ.

### FIELD OF THE INVENTION

The present invention relates to methods and compositions for producing cells resistant to apoptosis. More specifically, the genetically engineered cells of the present invention contain an anti-apoptosis gene, which is operably linked to a promoter that is activated under hypoxic conditions.

### BACKGROUND OF THE INVENTION

Transplanted cells provide the potential for treating various diseases because of their ability to detect and respond to physiologically important substances in the host. Patients having disease as a result of the loss or deficiency of hormones, neurotransmitters, growth factors or other physiological substances are considered to be among those who would achieve significant benefits from transplant therapy.

Cell implantation therapy can provide substances to replace or supplement natural substances that, due to their insufficiency or absence, cause disease. Several hormones, growth factors and other substances have been identified and are discussed in, e.g., U.S. Pat. No. 5,800,828 (which is incorporated herein by reference), as potential therapeutics that could be administered to an individual using transplanted cells. For example, implantation of pancreatic islet cells could provide insulin as needed to a diabetic. Adrenal chromaffin cells or PC12 cells implanted in the brain may provide dopamine to treat patients with Parkinson's disease. Cell implantation therapy can also be used to supply biologically active molecules for the treatment or prevention of neurodegenerative conditions, such as Parkinson's disease, Huntington's disease, Alzheimer's disease, and Acquired Immune Deficiency Syndrome-related dementia. Additionally, lymphokines and cytokines may also be supplied to modulate the host immune system. Other biologically active molecules which may be provided by implanted cells include catecholoamines, endorphins, enkephalins, and other opioid or non-opioid peptides that are useful for treating pain. Enzymatic deficiencies may also be treated by cell implantation therapy. Alternatively, the biologically active molecule may remove or eliminate deleterious molecules from the host.

Because cells that are implanted may be foreign to the host, it is necessary to prevent the host immune system from attacking and thereby causing the death of the implanted cells. In addition, cells that secrete such therapeutic substances may have been derived from transformed cells or have been infected with viruses and may therefore present a potential threat to the host in the form of increasing the likelihood of tumor formation. A method that has been used to protect the transplanted cells from host immune response involves the encapsulation of cells in a device that effectively isolates the implanted cells from the immune system. A common feature of isolation devices is a colony of living cells surrounded by a permeable membrane. The transport of oxygen, nutrients, waste, and other products across the membrane is driven by pressure and/or diffusion gradients.

If insufficient transport of these substances is provided for either the number or volume of cells, cell viability and function may be diminished. One critical element has been the level of oxygen reaching the cells. For example, immunoisolated devices have been demonstrated to diminish islet oxygenation in vitro. Schrezenmeir et al., Horm. Metab. Res. 25: 204-209 (1993). Indeed, hypoxia has been suggested as the major reason for the vulnerability of transplanted islets in the first few days of engraftment. Davalli et al., Diabetes 45: 1161-1167 (1996).

This oxygenation problem is compounded by the design of the encapsulation devices themselves. Although the semipermeable membrane allows free diffusion of oxygen, an oxygen gradient may form depending on the packing density of the cells inside the device. As cell density within the device increases, less oxygen becomes available for cells on the interior of the device. When too many cells are consuming oxygen within the device, the local concentration drops below the minimum level require for cell viability. Cells that are located near the outer surface of the cell mass will typically receive ample oxygen, while cells located in the interior will be the first to die or otherwise become disabled. Cell masses which become too large may inhibit diffusion of nutrients or gasses into the depths of the cell mass, resulting in the death of such cells and a correspondingly decreased substance output. This phenomenon is reported by Schrezenmeir et al., Transplantation Proceedings, 24: 2925-2929 (1992), which report a central core of necrosis in islets greater than about 150 microns diameter after culturing of the islets in an encapsulating device. Additionally, the secretion of other factors associated with lysis of dead cells may be harmful to the host or adjacent cells.

One method for maintaining the viability of cells within an encapsulating device is to make the device sufficiently narrow to keep the cells sufficiently close to the permeable membrane in contact with the environment. For instance, devices are often shaped to maximize surface area to volume rations. These devices are generally flat sheet devices or tubular hollow fiber membranes. Additionally, because of the oxygen gradient, the cell packing density is limited. Decreasing the device diameter and cell density however, can be problematic where a large number of cells is required in order to obtain an adequate therapeutic dose. For example, when treating diabetes, a vast amount of β-cells must be implanted into the host in order to achieve an efficacious result with β-cell transplantation. According to the International Islet Transplant Registry, at least 6000 human islet equivalent per kilogram of body weight (“IEQ/Kg”) of recipient are necessary to achieve normoglycemia in a type I diabetic patient. Thus, the encapsulation devices must be large enough to support this number of cells but cannot be loaded at a density that would limit oxygenation of the cells.

Therefore, a need remains in the art for hypoxia resistant and/or apoptosis resistant cells which could be packed more densely and which would be ideal for encapsulated cell therapy.

### SUMMARY OF THE INVENTION

The present invention relates to cells genetically engineered to contain an anti-apoptosis gene operably linked to a promoter that is activated under hypoxic conditions. The preferred anti-apoptosis gene is the bcl-2 gene encoding for the bcl-2 protein. The term “bcl-2 protein” is used herein as a generic term to refer to native protein, biologically-active fragments, muteins or analogs of bcl-2, preferably human or murine bcl-2. Alternative preferred anti-apoptosis genes include genes encoding for the native protein, biologically-active fragment, or mutein or analog of bfl-1, bclX_{L}, Bax, Mcl-1, A1, ced-9, LMW5-HL, and BHRF-1. Promoters that are activated under hypoxic conditions include promoters for phosphoglycerate kinase (“PGK”), hsp 70 and fos.

In a preferred embodiment, the cells of the present invention contain the human bcl-2 anti-apoptosis gene operably linked to the PGK promoter. Preferably, the cells transformed with the bcl-2-PGK construct are either βTc-tet cells or muscle fibroblasts. In a most preferred embodiment, the bcl-2 gene is also operably linked to a sequence (approximately 500 bp) of the woodchuck hepatitis virus (“WHV”) known to enhance expression.

The cells of the present invention may be implanted directly into the patient. Preferably, however, the cells are encapsulated in a semipermeable immunoisolatory membrane to form bioartificial organs.

The cells of the present invention can be used for treating a variety of diseases, including insulin-dependent diabetes mellitus. The cells of the present invention can be implanted either encapsulated or unencapsulated.

### BRIEF DESCRIPTION OF THE DRAWINGS

FIG. 1 is a schematic representation of the of the vector used to express hbcl-2 in β-cells, wherein 5′LTR indicates the coding sequences for the long terminal repeat of retrovirus; SD and SA indicates the coding sequences for the splice donor and acceptor; Psi indicates the coding sequences for the extended packaging signal; RRE indicates the coding sequences for the rev response element; PGK indicates the coding sequences for the mouse phosphoglycerate kinase promoter; hBCL-2 indicates the coding sequences for human bcl-2 cDNA; WHV indicates the sequence from the Woodchuck Hepatitis Virus, and 3′ΔU3LTR indicates the coding sequences for the long terminal repeat with an internal deletion in the U3 promoter/enhancer region.

FIG. 2 is a diagrammatic representation of a Western blot analysis of bcl-2 in infected cells wherein cellular extract of indicated β-Tc-tet cells were subjected to SDS/PAGE, immunoblotted, probe with an anti-Tag antibody, and reprobed with the anti-hbcl-2 antibody. In Panel A, cells were grown without tetracycline for the same number of passage. In Panel B, βTc-Tet-bcl-2 cells were growth arrested by 1 μg/ml tetracycline for the indicated number of days before protein extraction. MW indicates molecular weight markers.

FIG. 3 is a line graph depicting the time course of cell viability of proliferating control lacZ cells (dashed lines) and bcl-2 cells (solid lines). The number per random field of dead (triangles), apoptotic (circles), and viable (squares) cells were determined and are expressed as the mean±SE of triplicate determinations.

FIG. 4 is a line graph depicting the time course of cell viability of growth-arrested control lacZ cells (dashed lines) and bcl-2 cells (solid lines) following 500 nM staurosporine treatment. Cells were growth arrested in tetracycline for one week before staurosporine treatment. The number per random field of viable (squares) and dead or apoptotic (triangles) cells were determined and are expressed as the mean±SE of triplicate determinations.

FIG. 5 is a diagrammatic representation of a Western blot analysis showing DNA fragmentation in control lacZ cells and bcl-2 cells treated with 0, 250, or 500 nM staurosporine. MW indicates molecular weight markers.

FIG. 6 is a bar graph depicting cytokine-induced apoptosis in βTc-tet cells.

FIG. 7 is a bar graph depicting apoptosis of conditionally-immortalized βTc-tet cells following growth arrest by incubation for 72 hrs in the presence of tetracycline. The number of viable or apoptotic and necrotic cells was determined in random fields and expressed as the mean±SE of triplicate determinations.

FIG. 8 is a bar graph comparing the apoptotic response to hypoxia of proliferating and growth arrested (TC) control βTc-tet cells (Ct) and bcl-2 cells (Bcl-2). Duplicate 12-well plates were incubated under hypoxic conditions (2.6% O₂) for 24 hrs. Apoptosis/viability ratios were determined by scoring random fields of cells for condensed and fragmented nuclear morphology with the Hoechst/PI staining procedure.

FIG. 9 is a bar graph comparing the apoptotic response to hypoxia of proliferating control lacZ cells (Ct) and bcl-2 cells (Bcl-2) incubated under hypoxic conditions (2.6% O₂) for 16 and 24 hrs. Apoptosis/viability ratios were determined by scoring random fields of cells for condensed and fragmented nuclear morphology with the Hoechst/PI staining procedure.

FIG. 10 is a bar graph comparing the density-dependent glucose-stimulated insulin secretion of control βTc-tet cells (Ct, open bars) and bcl-2 cells (Bcl-2, closed bars) encapsulated in 1% agarose. Clusters of βTc-tet cells were prepared and embedded at the indicated cell density into 1% agarose capsules. Following three weeks of culture, the amount of insulin secreted in one hour was determined using RIA. Results shown are the mean±SE of four independent experiment done in triplicate. Control cells were already showing maximal secretion at 1×10⁶ cells/ml FIGS. 11 and 12 are line graphs depicting the time course of glucose-induced insulin secretion by control βTc-tet cells and Bcl-2 transformed cells, respectively. Cells were perfused with a Krebs buffer solution containing 2.8 or 16.7 mM glucose, as indicated. Data are expressed as mean±SE for three perfusions.

FIG. 13 is a bar graph comparing the glucose-induced secretion by control βTc-tet cells (open bars) and Bcl-2 cells (hatched bars) incubated for three hours in Krebs buffer containing 2.8 or 16.7 mM glucose, as indicated, under normoxic (four leftmost bars) or hypoxic (2.6% O₂) conditions (four rightmost bars). Data are expressed as mean±SE for two independent secretions done in triplicates. Statistical differences between groups was determined using Student's paired or unpaired t-test.

FIG. 14 is a line graph depicting blood glucose levels of diabetic mice implanted with 2×10⁶ control βTc-tet cells (dashed line) or Bcl-2 cells (solid line) under the kidney capsule. Blood glucose levels were measured every two days and a slow-release tetracycline pellet was implanted when glycemia fell below 3 mg/ml. Data are expressed as the mean±SE (n=8-10). The difference between the two groups at the 75-day time point is significant by the t-test (p≦0.05).

### DETAILED DESCRIPTION OF THE INVENTION

The present invention provides methods of producing genetically-engineered cells containing an anti-apoptosis gene, which is operably linked to a promoter that is activated under hypoxic conditions. Any suitable cell may be transformed with the recombinant DNA molecules of the present invention. For example, if the cells are used to treat IDDM, the cells should produce insulin in response to blood glucose levels. These insulin-secreting cells may be genetically engineered for this phenotypic trait or this phenotypic trait may be endogenous to the cell line that is selected. β-cells or islets are but one example of the latter cell type suitable for use in the present invention.

Any gene encoding a polypeptide or protein with anti-apoptotic properties may be used in the present invention. The preferred anti-apoptosis gene is the bcl-2 gene. The bcl-2 protein is a 26 kD membrane-associated cytoplasmic protein. See, e.g., U.S. Pat. No. 5,202,429, incorporated herein by reference. The capacity of bcl-2 to enhance cell survival has been reported to be related to its ability to inhibit apoptosis initiated by several factors, such as cytokine deprivation, radiation exposure, glucocorticoid treatment, and administration of anti-CD-3 antibody. Nunez et al., J. Immunol. 144: 3602 (1990); Hockenbery et al., Nature 348: 3602 (1990); Vaux et al., Nature 335: 440 (1988); Alnemri et al., Cancer Res. 52: 491 (1992); Sentman et al., Cell 67: 879 (1991); Strasser et al., Cell 67: 889 (1991). Upregulation of bcl-2 expression has also been reported to inhibit apoptosis of EBV-infected B-cell lines. Henderson et al., Cell 65: 1107 (1991). The expression of bcl-2 has also been shown to block apoptosis resulting from expression of the positive cell growth regulatory proto-oncogene, c-myc, in the absence of serum or growth factors. Wagner et al., Mol. Cell. Biol. 13: 2432 (1993).

A family of bcl-2-like genes have been identified and evidence indicates that they participate in regulating cell death. Other proteins which interact with and/or are structurally related to the bcl-2 gene product include, for example, bfl-1 (Shin et al., U.S. Pat. No. 5,843,773); bclX_{L} and bclX_{S} (Boise et al., (1993) Cell 74: 597; Gonzalez-Garcia et al., (1994) Development 120: 3033; Gottschalk et al., (1994) Proc. Natl. Acad. Sci. USA 91: 7350); Bax (Oltvai et al., (1993) Cell 74: 609); Mcl-1 (Kozopas et al., (1993) Proc. Natl. Acad. Sci. USA 90: 3516); and A1 (Lin et al., (1993) J. Immunol. 151: 179). The family of bcl-2-related proteins also includes the nematode protein ced-9 (Vaux et al, (1992) Science 258: 1955; Hengartner et al., (1992) Nature 356: 494; Hengartner M O and Horvitz H R (1994) Cell 76: 665) and two DNA virus proteins, LMW5-HL and BHRF-1 of the Epstein Barr Virus.

Similarly, any suitable promoter that is activated under hypoxic conditions can be used in the construct of the present invention. In a particularly preferred embodiment of the present invention, a vector is constructed to contain the bcl-2 anti-apoptosis gene operably linked to the mouse phosphoglycerate kinase promoter (“the PGK promoter”) which is activated under hypoxic conditions. Thus, when the cells of this embodiment experience hypoxic conditions, the PGK promoter activates transcription of the bcl-2 gene, resulting in an expression of the bcl-2 protein. The expression of the bcl-2 protein inhibits apoptosis and allows the cells to continue to survive in the hypoxic environment. Preferably, this vector includes an approximately 500 bp DNA sequence from the Woodchuck Hepatitis Virus (“WHV”). This sequence greatly stabilizes the unspliced mRNAs and enhances translation efficiency resulting in approximately 5 to 10 fold increase in expression in cells exposed to hypoxic conditions as compared to normoxic conditions.

Any suitable viral vector which can effectively transform β-cells is contemplated by the present invention. Lentiviral vectors, however, are generally preferred. To access whether lentiviral vectors can effectively transform β-cells, a lentiviral vector in which the E. Coli lacZ gene is driven by an internal mouse phosphoglycerate-kinase promoter was used to transform the βTc-tet murine cell line. This cell line has a unique cell cycle control imposed by the large T antigen gene, which is operably linked to a tetracycline (“tet”) promoter.

The tet promoter can be constructed so that it either positively or negatively activates the large T antigen. Thus, in one embodiment, in the presence of tetracycline, the large T antigen is activated, of which holds the cell in the S-phase of the mitotic cycle (hereinafter “growth arresting”). In another preferred embodiment, in the absence of tetracycline, the large T antigen is activated and the cell is held in the S-phase of the mitotic cycle. Activation of the tet promoter does not affect the bcl-2 gene and activation of the bcl-2 gene via hypoxic conditions does not affect the tet promoter system.

The cell growth arresting system allows the regulation of growth of the βTc-tet cells. This feature is important in two contexts. First, cell growth can be controlled during cell culturing. Second, the density of cells within the implanted device can be controlled.

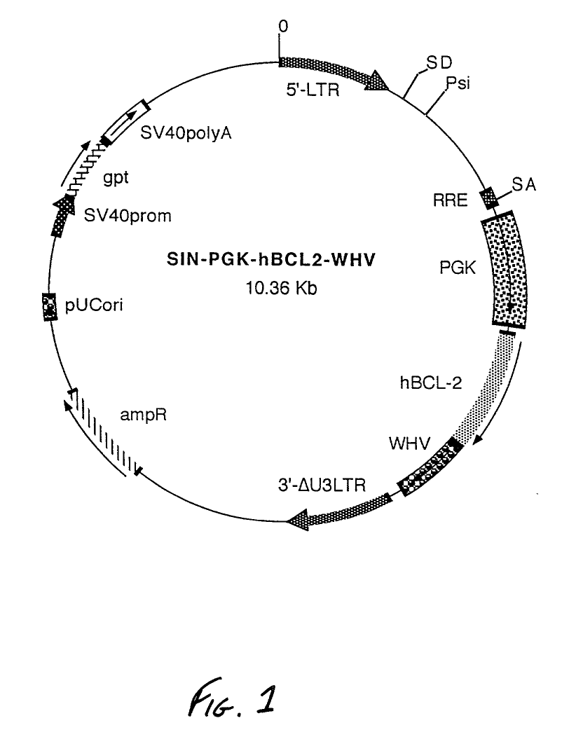

In one embodiment of the present invention, βTc-tet cells were transformed with a lentiviral vector containing the bcl-2 gene operably linked to the PGK promoter and to the expression enhancing sequence of the WHV. The vector has a 5′long terminal repeat (“LTR”), a splice donor (“SD”), splice acceptor (“SA”) and an extended packing signal (“Psi”). The vector also contains a Rev. response element (“RRE”) and a PGK promoter operably linked to the hBcl-2 gene, which in turn is operably linked to the WHV expression enhancing sequence from the woodchuck hepatitis virus and a 3′-ΔU3LTR, with an internal deletion in the Us promoter/enhancer region. The vector also contains an ampicillin resistance AMPR gene and a PUC origin. The gpt gene and the SV40 polyA tail. The Vector is approximately 10.36 kb. A schematic illustration of the vector is shown in FIG. 1.

A control group of cells was created by transforming βTc-tet cells with the lacZ gene. Transfections were done on growth-arrested cells as well as non-growth-arrested cells, with similar infection rates. More than 98% of the cells transfected with the bcl-2 gene stained strongly positive for bcl-2, with a typical mitochondrial and perinuclear granular immunofluorscence. Interestingly, after a few passages, the morphology of the culture changed with the bcl-2 cells appearing more flat and able to grow at a much higher density than the control cells. Expression of bcl-2 was confirmed by western blot analysis.

Bcl-2 transformed cells were next tested for resistance to apoptosis. Apoptosis was induced in several ways. First, apoptosis was induced by exposing the cells to staurosporine, a known apoptosis inducer. Cells were also exposed to hypoxia which also resulted in apoptosis. Lastly, growth arrest of the βTc-tet cells induced apoptosis, a method also known in the art. Regardless of the method used to induce apoptosis, the bcl-2 transformed cells showed a dramatic resistance to apoptosis as indicated by DNA fragmentation, the hallmark of apoptosis, and by histology. Resistance was conferred to cells regardless of whether they were growth arrested or not.

In addition to measuring cell viability, insulin secretion of bcl-2 transformed βTc-tet cells and wild-type βTc-tet cells was measured for both basal insulin secretion and insulin secretion in response to increased levels of glucose. Apoptosis was induced in these cells by exposing them to 2.3% O₂ for three hours. These hypoxic conditions were mild enough not to affect cell viability. Under these conditions, the wild-type βTc-tet cells showed an approximately 2-fold decrease in basal insulin secretion as compared to normoxic conditions, while the bcl-2 cells were almost completely protected. Additionally, glucose-stimulated insulin secretion was 61% under hypoxic conditions for the wild-type cells, while the bcl-2 transformed cells retained 82% of their insulin secretion. Thus, under these mild hypoxic conditions, the bcl-2 transformed cells retained their basal insulin secretion levels and suffered only modest effects to their glucose-stimulated insulin secretion.

Bcl-2 transformed cells were also encapsulated in 1% agarose to simulate conditions in cell encapsulation devices. Under these conditions, wild-type β cells were barely able to survive cell densities above 1×10⁶ cells/ml and good survival was still detected up to 20×10⁶ cells/ml. Additionally, at the same cell density in agar, bcl-2 expressing cells secreted approximately 60-fold more insulin in 24 hours than did the wild-type cells.

The cells of this invention may be implanted into a mammal, including a human, for the treatment of IDDM. The cells may be implanted as either encapsulated or unencapsulated. If unencapsulated cells are implanted, any suitable implantation protocol may be used. Preferably, however, the cells are encapsulated in a semipermeable membrane prior to encapsulation. If the cells are encapsulated as such, they are referred to here, and in the literature in general, as bioartificial organs (“BAO”).

BAOs may be designed for implantation into a host or can be made to function extra-corporeally. The BAOs useful in the present invention typically have at least one semipermeable membrane or jacket surrounding a cell-containing core. The jacket permits diffusion of nutrients, biologically active molecules, and other selected products to cross the membrane into and out of the cell-containing core. Preferably, if the BAO is to be implanted into the host, the membrane is biocompatible, such that it does not have a substantial deleterious effect on the host upon implantation.

Preferably, the membrane is also immunoisolatory, so as to isolate the cells from the cellular and molecular effectors of immunological rejection. Use of an immunoisolatory membrane allows for the transplantation of allogenic and xenogenic cells into a host without the use of immunosuppressive drugs. If biologically active molecules are released from the immunoisolated cells, the molecules diffuse from the cell-containing core through the membrane and into the host's body. If metabolic functions are provided by the isolated cells, the metabolites diffuse across the membrane and can be metabolized by the cells in the cell-containing core.

A variety of types of membranes have been used in the construction of BAOs. Generally, the membranes used in BAOs are either microporous or ultrafiltration grade membranes. A variety of membrane materials have been suggested for use in BAOs, including PAN/PVC, polyurethanes, polysufones, polyvinylidienes, and polystyrenes. Typical membrane geometries include flat sheets, which may be fabricated into “sandwich” type constructions, having a layer of living cells positioned between two essentially planar membranes with seals formed around the perimeter of the device. Alternatively, hollow fiber devices may be used, where the living cells are located in the interior of a tubular membrane. Hollow fiber BAOs may be formed step-wise by loading living cells in the lumen of the hollow fiber and providing seals on the ends of the fiber. Hollow fiber BAOs may also be formed by a coextrusion process, where living cells are coextruded with a polymeric solution which forms a membrane around the cells.

The encapsulating membrane of the BAO may be made of a material which is the same as that of the core, or it may be made of a different material. In either case, a surrounding or peripheral membrane region of the BAO which is permselective and biocompatible will be formed. The membrane may also be constructed to be immunoisolatory, if desired. The core contains isolated cells, either suspended in a liquid medium or immobilized within a hydrogel matrix.

The choice of materials used to construct the BAO is determined by a number of factors. Various polymers and polymer blends can be used to manufacture the capsule jacket. Polymeric membranes forming the BAO and the growth surfaces therein may include polyacrylates (including acrylic copolymers), polyvinylidenes, polyvinyl chloride copolymers, polyurethanes, polystyrenes, polyamides, cellulose acetates, cellulose nitrates, polysulfones, polyphosphazenes, polyacrylonitriles, poly (acrylonitrile/covinyl chloride), as well as derivatives, copolymers and mixtures thereof. Devices and methods for constructing same are disclosed, e.g., in U.S. Pat. Nos. 5,800,828; 5,653,975; and 5,873,234, each incorporated herein by reference.

The jacket of the BAO will have a pore size that determines the nominal molecular weight cut off (nMWCO) of the permselective membrane. Nominal molecular weight cut off is defined as 90% rejection under convective conditions. In situations where it is desirable that the BAO is immunoisolatory, the membrane pore size is chosen to permit the particular factors being produced by the cells to diffuse out of the vehicle, but to exclude the entry of host immune response factors into the BAO. Typically the nMWCO ranges between 50 and 2000 kD, preferably between 80 and 600 kD, most preferably between 90 and 180 kD. The most suitable membrane composition will also minimize reactivity between host immune effector molecules known to be present at the selected implantation site, and the BAO's outer membrane components.

The core of the BAO is constructed to provide a suitable local environment for the particular cells isolated therein. The core can comprise a liquid medium sufficient to maintain cell growth. Alternatively, the core can comprise a gel matrix. The gel matrix may be composed of hydrogel (alginate, “Vitrogen™”, etc.) or extracellular matrix components.

Compositions that form hydrogels fall into three general classes. The first class carries a net negative charge (e.g., alginate). The second class carries a net positive charge (e.g., collagen and laminin). Examples of commercially available extracellular matrix components include Matrigel™ and Vitrogen™. The third class is net neutral in charge (e.g., highly crosslinked polyethylene oxide, or polyvinylalcohol).

Any suitable method of sealing the BAO may be used, including the employment of polymer adhesives and/or crimping, knotting and heat sealing. These sealing techniques are known in the art.

One or more in vitro assays are preferably used to establish functionality of the BAO prior to implantation in vivo. Assays or diagnostic tests well known in the art can be used for these purposes. See, e.g., Methods In Enzymology, Abelson [Ed], Academic Press, 1993. For example, an ELISA (enzyme-linked immunosorbent assay), chromatographic enzymatic assay, or bioassay specific for the secreted produce can be used. If desired, secretory function of an implant can be monitored over time by collecting appropriate samples (e.g., serum) from the recipient and assaying them. If the recipient is a primate, microdialysis may be used.

The number of BAOs and BAO size should be sufficient to produce a therapeutic effect upon implantation is determined by the amount of biological activity required for the particular application. In the case of secretory cells releasing therapeutic substances, standard dosage considerations and criteria known to the art are used to determine the amount of secretory substance required.

Implantation of the BAO is generally performed under sterile conditions. Typically, the BAO is implanted at a site in the host which will allow appropriate delivery of the secreted produce or function to the host and nutrients to the encapsulated cells or tissue, and will also allow access to the BAO for retrieval and/or replacement. The preferred host is a primate, most preferably a human.

This invention contemplates implantation into the kidney subcapsular site, and intraperitoneal and subcutaneous sites, and other therapeutically beneficial sites.

In order that this invention may be better understood, the following examples are set forth. These examples are for purposes of illustration only, and are not to be construed as limiting the scope of this invention in any manner.

### EXAMPLE 1

Preparation of Lentiviral Vectors and Transformation of βTc-tet Cells

Cells were grown in Dulbecco's modified Eagles medium (DMEM, Gibco) containing 24 mM glucose and supplemented with 15% horse serum (Amimed), 2.5% fetal bovine serum (Gibco), 100 U per ml penicillin, and 100 μg per ml streptomycin. Growth arrest was induced by including 1 μg/ml tetracycline (Fluka). Clusters were generated on rotative culture in 100 mm bacterial petri dishes.

The EcoRl bcl-2 cDNA was subclone first into pSP72 (Promega). It was then inserted as a BgIII-XhoI fragment into the SIN-PGK-WHV vector. High-titer stocks of lentiviral vectors carrying the PGK driven lacZ or bcl-2 genes packaged by the multiply attenuated lentivirus CMVΔRS.91, and pseudotyped with the VSV-G envelope (plasmid pMD-G) were prepared by transient transfection of 293T. Viral stocks of lacZ virus were tested on human 293T or rat 208F fibroblasts in six serial dilutions of viral stock (5 fold to 3125 fold) and the viral titer was determined by counting the number of foci of X-Gal-containing blue cells per well divided by the dilution factor. Bcl-2 virus titer was determined on serial dilutions of infected βTc-tet, and 208F cells using indirect immunofluorescence staining for bcl-2 protein. Pure viral stocks were tested for the presence of RCVs using the Hela-p4 assay. In addition, target cells were co-cultivated Hela-p4 cells for one month and thereafter the hela p4 cells were tested for the presence of β-Galactosidase enzymatic activity. None of these tests revealed the presence of tat-tranducing activity above background level. Murine leukemia virus vectors were prepared as described using the Phoenix-eco packaging cell line transiently transfected with a Babe-lacZ-puro vector.

A stock of the bcl-2 expressing viral vector was used to transform fresh βTc-tet cells at an MOI of 20 and following a one week recovery period, infected cells were plated on a glass-coverslip to detect by immunofluorescence the expression of the bcl-2 protein. As expected, more than 98% of the cells transfected with the bcl-2 gene stained strongly positive for bcl-2, with a typical mitochondrial and perinuclear granular immunofluorescence (data not shown).

As a control, replicating βTc-tet cells (doubling-time at passage 34=80 hrs) were also plated at a similar density and both cultures were transformed with a stock of lentiviral vector. The viral stock gave a titer of 1×10⁶ TFU/ml on 293T cells but transformed a maximum of 5-10⁴ proliferating βTc-tet cells in six well plates. Almost the same number of cells were infected both in the growth arrested and proliferating plates. These results demonstrated that lentiviral vectors can efficiently transform post-mitotic β-cells without apparent cytotoxicity. The viral vector was, however, less efficient at transforming the mouse beta cell-line than the human 293T cells, showing a 20 fold lower infectivity than in the human embryonic kidney-derived cell line. Murine leukemia virus vectors (MLV) could transform on 2 to 5% of proliferating cells. This result is not unexpected because only cells that go through the S-phase of the cell cycle in the 16 hrs following infection will be infected by oncoretroviruses. Growth-arrested cells were not transformed by MLV virus.

To measure expression of the protein of interest, cells were lysed in 1% Triton X100, 0.15M NaCl, and 10 mM Tris (pH7.4) with 50 μg/ml PMSF and Aprotinin at 4°C. for 30 minutes. Western-blot was performed using a mouse monoclonal anti-human bcl-2 protein (Boehringer) diluted 1/100e and a rabbit polyclonal anti-SV40 Tag antibody diluted 1/2000e. The bcl-2 antibody was used for immunofluorescence staining diluted 1/100e. Immunofluorescence staining after a few passages revealed an homogeneous cell population expressing very high levels of the anti-apoptotic protein bcl-2, which was easily confirmed by western-blot analysis.

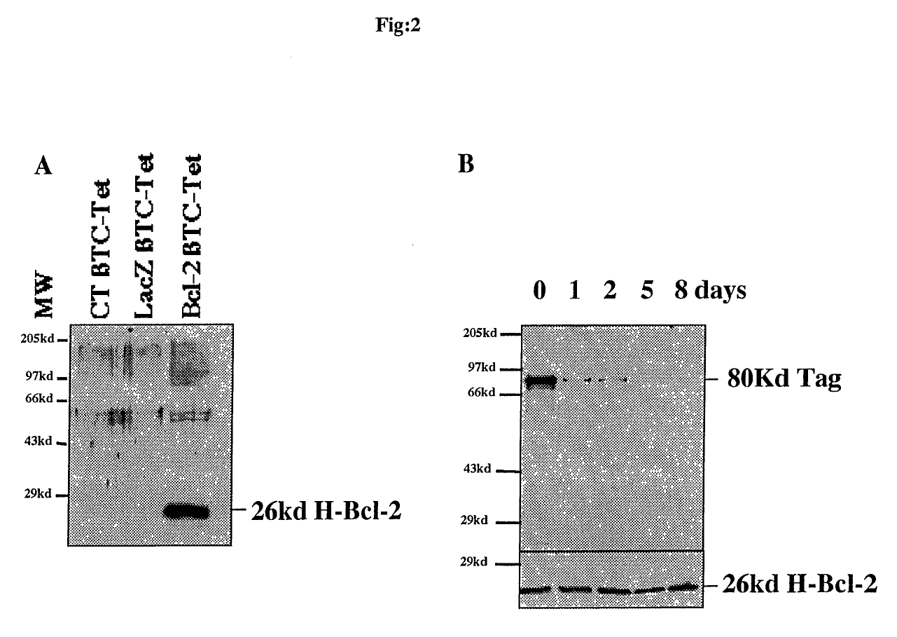

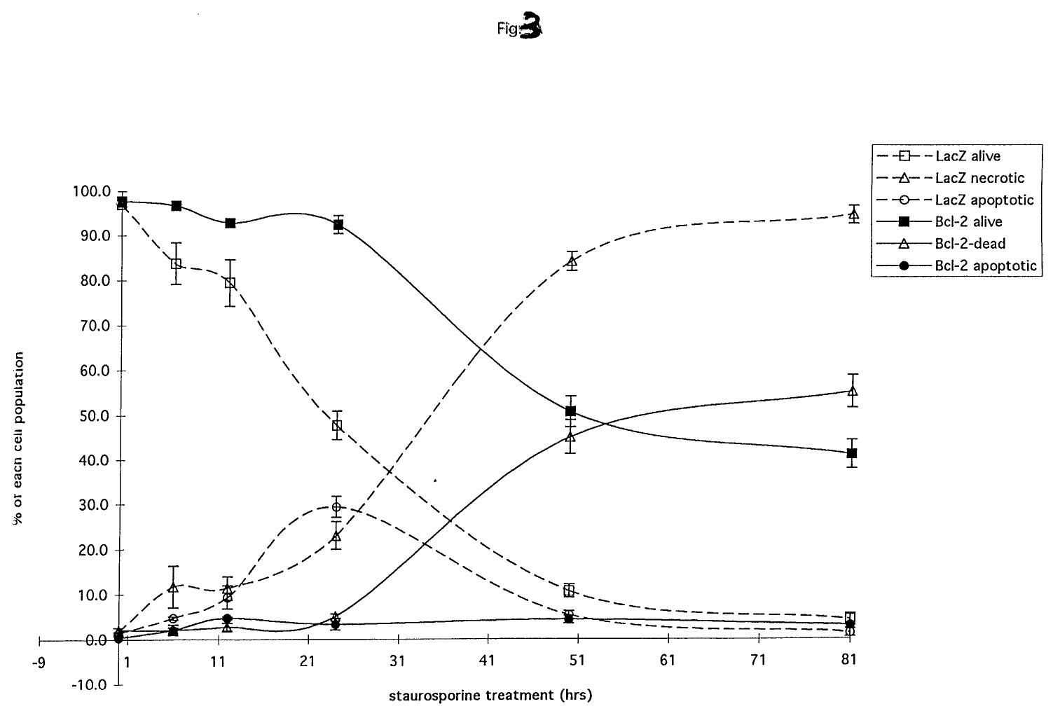

As illustrated in FIG. 2A, neither the control βTc-tet cells nor the lacZ transformed population of βTc-tet cells express the bcl-2 protein. In contrast, the bcl-2 transformed population showed strong expression (FIG. 2A). Following growth-arrest by exposure to tetracycline, the expression of the transformed bcl-2 was shown to be stable and apparently did not affect the shut-down of SV40 T antigen expression upon tetracycline administration. Confluent growth-arrested plates of bcl-2 cells were kept in culture for a period for several months without any sign of proliferation (data not shown). Stable and sustained expression of the transduced bcl-2 gene was followed for more than 10 months.

FIG. 2A shows cells which were grown at the same passage number and were not growth arrested with tetracycline. FIG. 2B shows β-TC-tet cells that have been transformed with bcl-2. The cells were grown in six well plates and growth arrested with tetracycline at a 1 μg/ml concentration for the number of days indicated in FIG. 2B. The blot was probed with anti-Tag antibody (upper panel) and was probed with the anti-hBlc-2 antibody (lower panel). Molecular weight markers were also run and are indicated in the figure.

### EXAMPLE 2

Overexpression of Bcl-2 Protects β-Tc-tet Cells From Staurosporine-Induced Apoptosis

Twenty-four hours before induction of apoptosis, proliferating (group 1) or growth-arrested (group 2) lacZ and bcl-2 transformed β-cells were plated in poly-lysine coated 96 well microtiter plates at a cell density of 3×10³ cells/well. In group 1, cells were not growth arrested with tetracycline. In group 2, cells were growth arrested in tetracycline for 1 week before plating and tetracycline was kept in the culture medium throughout the experiment. After plating, the cell medium was changed the next morning and a time course of cell viability was assessed following addition of 500 nM staurosporine to the cells.

During the time course, cells were examined by nuclear staining (Hoechst/PI) and inverted fluorescence microscopy. In this staining technique, viable cells have nuclei which stain as large and diffuse blue. Apoptotic cells have nuclei which appear as condensed and fragmented and intense blue or pink. Dead cells have nuclei which appear as red nuclei. Thus, apoptotic index can be calculated by viewing a random field and counting the number of each type of nuclei in that field. This number was then plated versus time.

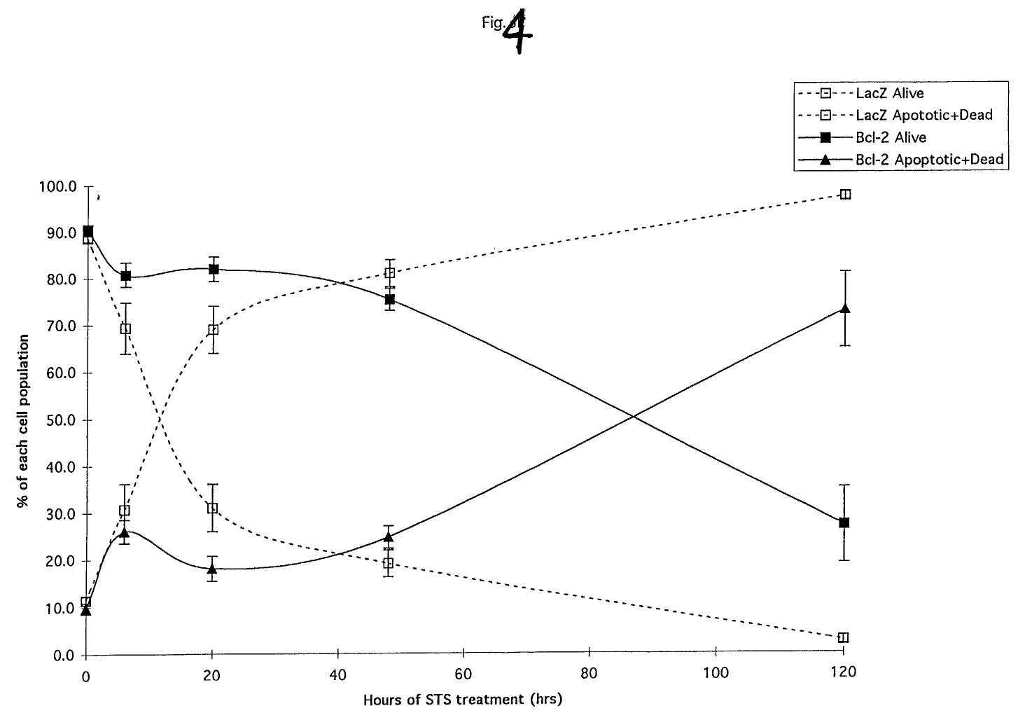

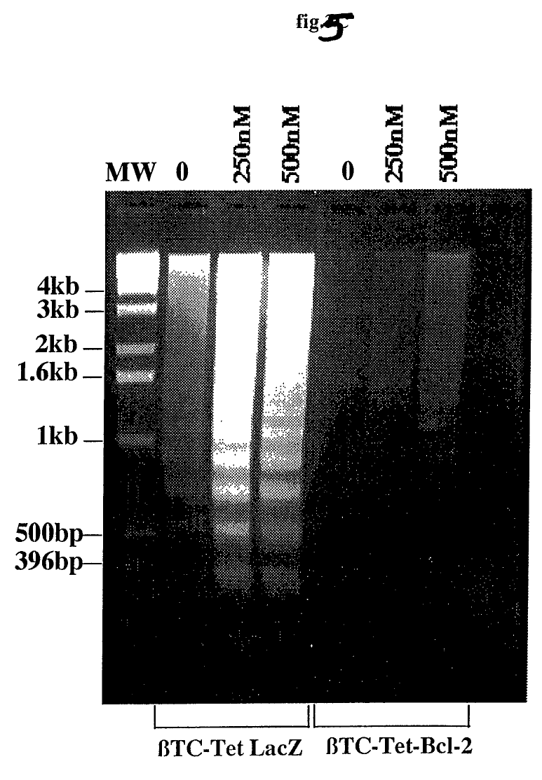

FIG. 3 depicts the time course of cell viability of lacZ and bcl-2 Tc-tet β-cells that were growth-arrested in tetracycline. The Bcl-2 βTc-tet cells were resistant to apoptosis induced by staurosporine, showing a delay in apoptosis, with many cells remaining viable after 72 hours. Conversely, the control lacZ βTc-tet cells were very sensitive to staurosporine, showing a dramatic increase in apoptosis after 24 hours (FIG. 3). As illustrated in FIG. 4, cells which were not growth-arrested showed a similar resistance to apoptosis.

In another experiment used to confirm staurosporine induced apoptosis, DNA fragmentation was analyzed. In this experiment, control lacZ βTc-tet cells and bcl-2 βTc-tet cells were growth arrested as described above. Cells were plated and exposed to staurosporine concentrations of 0, 250, and 500 nM. Following induction of apoptosis, cells 1×10⁶ were collected (both adherent and non-adherent) and lysed in 500 μl of hypotonic lysis buffer {10 mM Tris (pH 7.5), 20 mM EDTA, 0.5% Tx-100}. After 30 minutes incubation at 4°C., the supernatant was adjusted to 0.3M Na-acetate and extracted once with phenol-chloroform. The supernatant was treated with Dnase-free Rnase A (100 μg/ml) for 1 hour at 37°C., extracted with phenolchloroform, chloroform and precipitated with isopropanol. The resuspended DNA subjected to 2% agarose gel electrophoresis and stained with ethidium bromide.

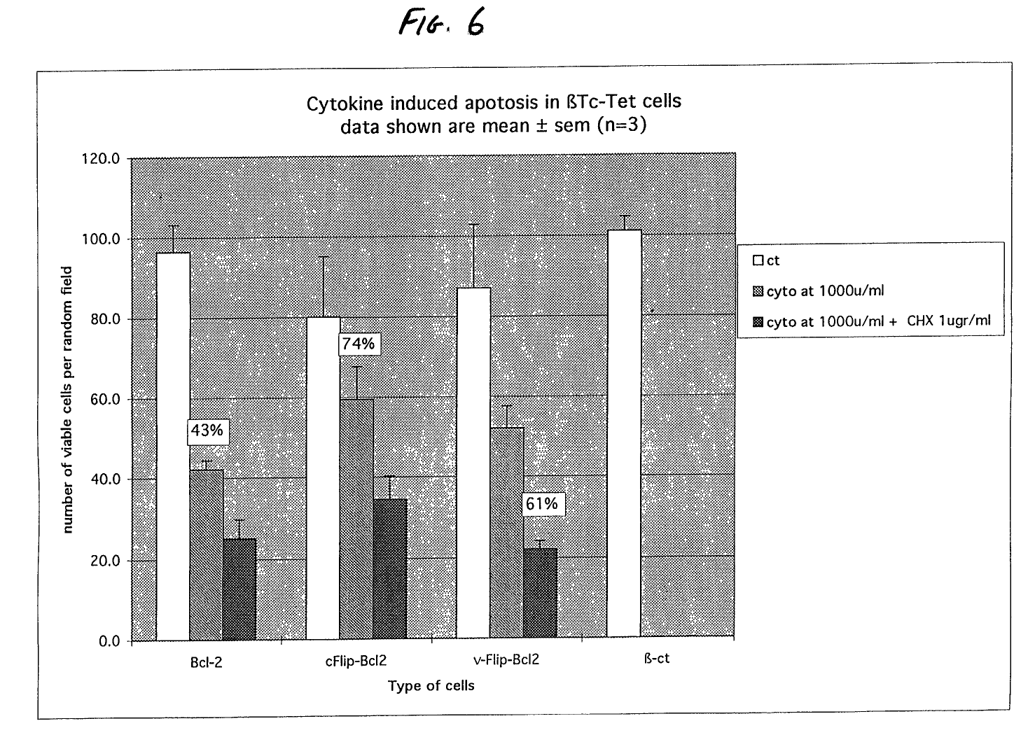

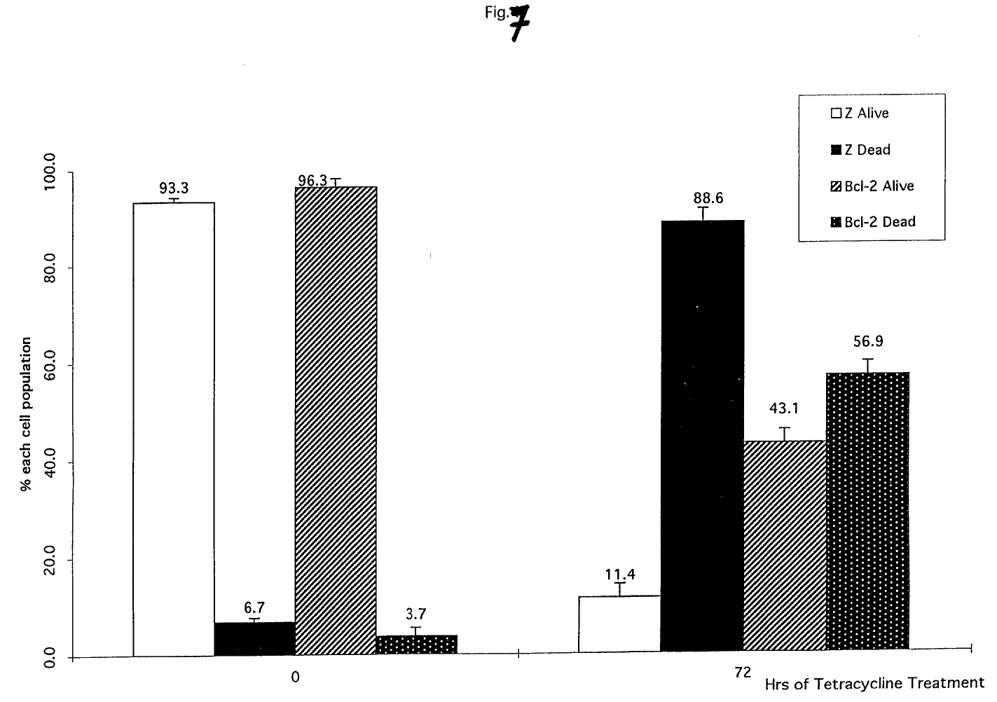

As seen in FIG. 5, control lacZ cells showed a dramatic amount of DNA fragmentation as indicated by the ladder effect on the gel. This effect was seen for both the 250 and 500 nM concentrations of staurosporine. In contrast, the bcl-2 cells exhibited no DNA fragmentation at either concentration of drug.

### EXAMPlE 3

Bcl-2 Transformed Cells Resist Apoptosis Induced by Growth Arresting the Cells

βTc-tet lacZ and bcl-2 cells were plated at 2.5×10⁶ cells per 100 mm dish. The cells were exposed to tetracycline for 72 hours and viability was determined as described above. As illustrated in FIG. 7, 89%±2.8% (n=3) of the lacZ cells undergo apoptosis compared to only 56%±1.8% (n=3) of the bcl-2 cells.

### EXAMPLE 4

Overexpression of Bcl-2 Protects βTc-tet Cells From Hypoxia-Induced Apoptosis

Duplicate 12-well plates of lacZ or bcl-2 transformed βTc-tet cells were plated at a density of 1×10⁵ cells/well and covered with a minimal amount of medium to reduce the possibility of oxygen gradient formation. Cells were exposed to hypoxia by placing them in a sealed Plexiglas chamber housed in an incubator at 37°C. Hypoxic conditions were achieved by continuously purging the chamber with humidified gas mixtures. Tested gas mixtures included (i) 5% CO₂, 95% N₂; (ii) 1.32% CO₂, 5% CO₂, and 93.68% N₂; (iii) 2.6% O₂, 5% CO₂, and 92.4% N₂; (iv) 5.32% O₂, 5% CO₂ and 89.68% N₂. Oxygen levels were continuously monitored using an oxygen electrode (Clark, OXI 3000™, WTW, Weilheim, Germany). Typically, equilibration from normoxic conditions to hypoxic levels of 5% O₂ and 1.3% O₂ took 2 min and 6 min, respectively. Following removal from the hypoxic chamber, the cells were immediately assayed. A control plate of βTc-tet cells was placed in a standard 37°C. incubator at approximately 21% O₂ with 5% CO₂, and 79% N₂.

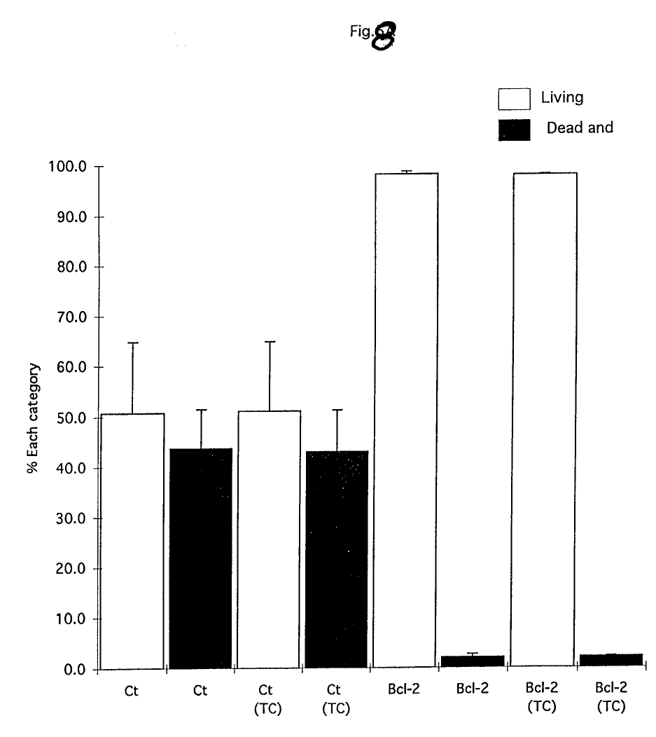

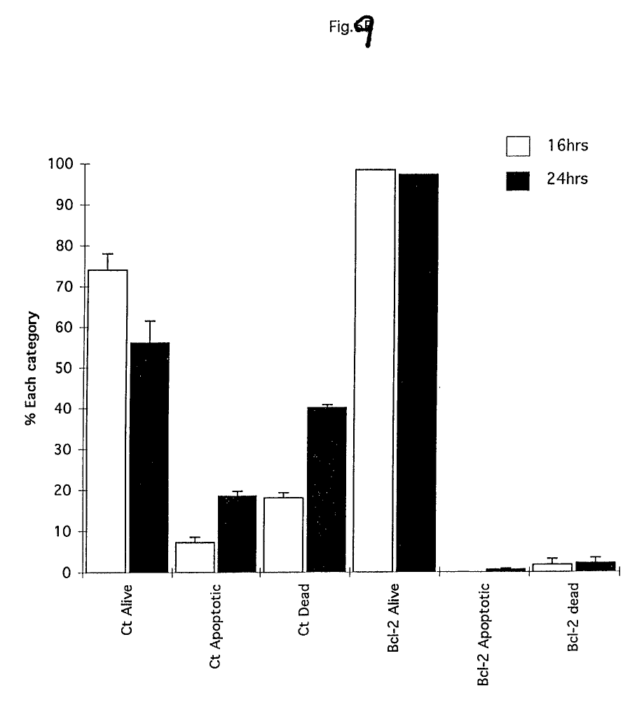

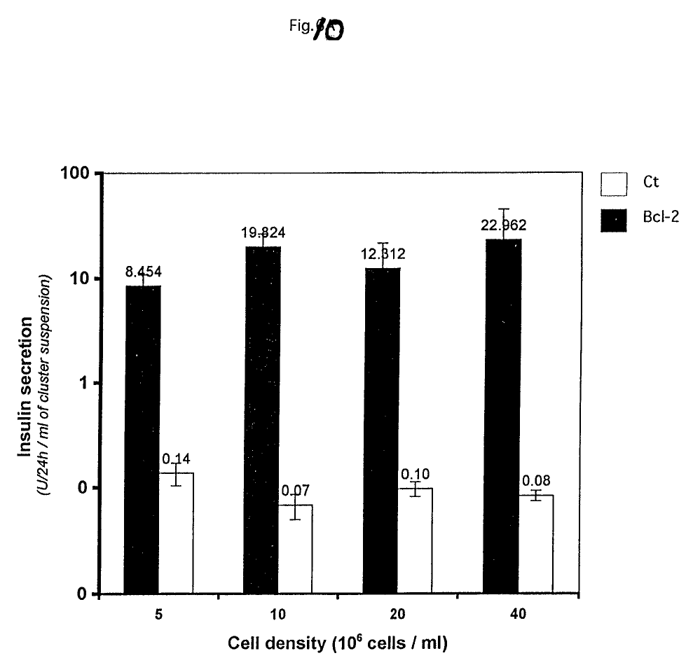

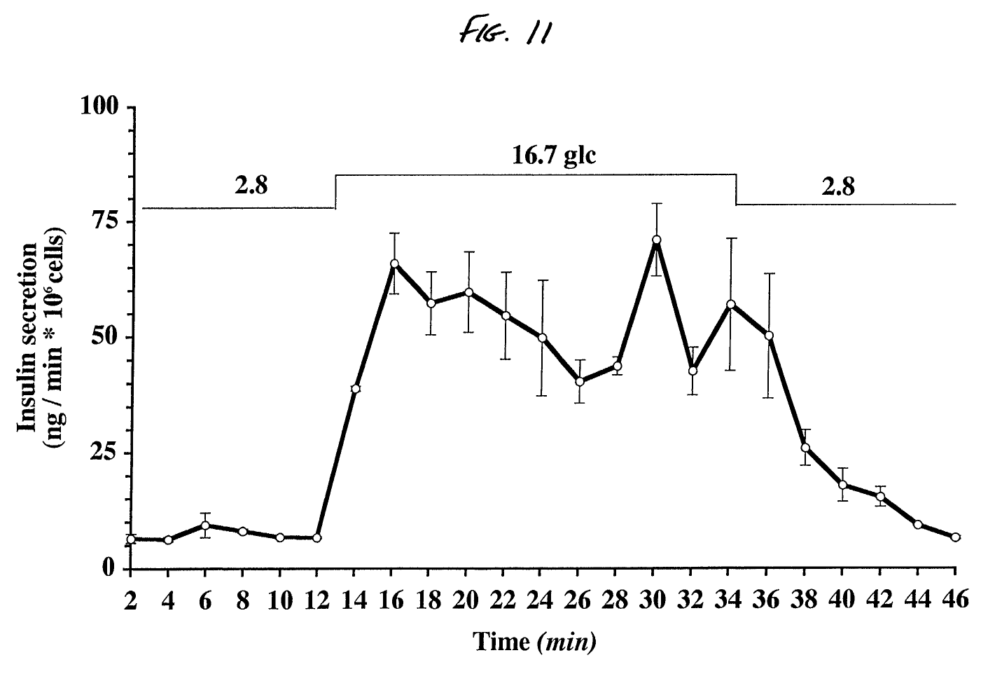

Under anoxic conditions, both the lacZ and the bcl-2 transformed cells showed 100% mortality. At 2.6% O₂, approximately 50%±7% of the control βTc-tet cells were dead after 24 hours, with many cells exhibiting nuclear condensation and fragmentation, whereas the bcl-2 transformed cells exhibited only a 2%±0.6% mortality rate. As depicted in FIG. 8, cells which were growth arrested as described above were protected almost equally as well.

In addition to measuring viability, insulin secretion under hypoxic conditions was also measured. Bcl-2 transformed and wild-type cells were plated in 12-well plates at a density of 10₅ cells/well 24 hours before incubation under hypoxic conditions. Cells were then incubated for 30 minutes in Krebs-Ringer bicarbonate, HEPES buffer (“KRBH”), pH 7.4, containing 1% bovine serum albumin and 2.8 mM glucose. After the 30 minute incubation, the cell medium was changed to KRBH containing either 2.8 mM glucose or 16.7 mM glucose, and 250 μm isobutyl methylxanthine (“IBMX”), which had been pre-equilibrated under the hypoxic conditions to be studied. The cells were incubated under hypoxic conditions (23% O₂) for three hours. Duplicate control plates of bcl-2 transformed and wild-type cells were incubated at normnoxic conditions under identical conditions.

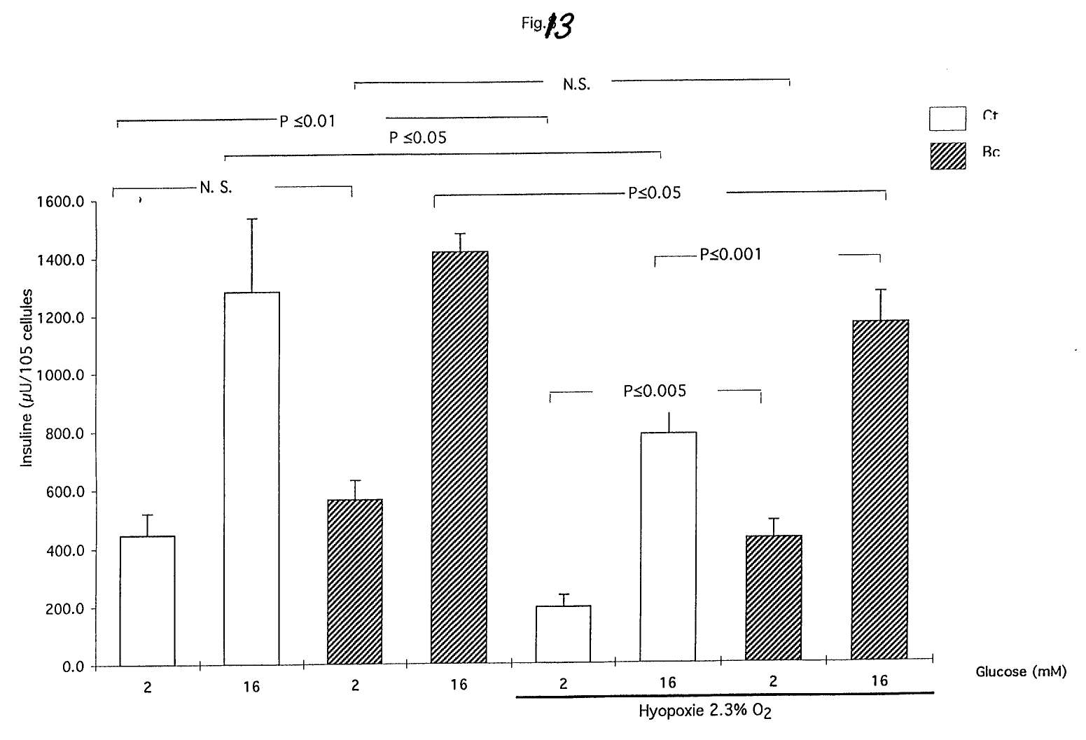

Exposure to 2.6% O₂ for three hours does not affect wild-type β-cell survival. As shown in FIG. 13, however, these wild hypoxic conditions induce an approximate two-fold decrease in basal insulin secretion in the wild-type cells (43% of normoxic conditions). Additionally, glucose stimulated insulin secretion was also significantly decreased by hypoxia to 61% of the normoxic level. In contrast, bcl-2 expressing cells showed similar basal insulin secretion under hypoxic and normoxic conditions (FIG. 13). Additionally, stimulated insulin secretion under hypoxic conditions was 82% of normoxic conditions.

### EXAMPLE 5

Bcl-2 Transformed Cells Encapsulated in Agarose

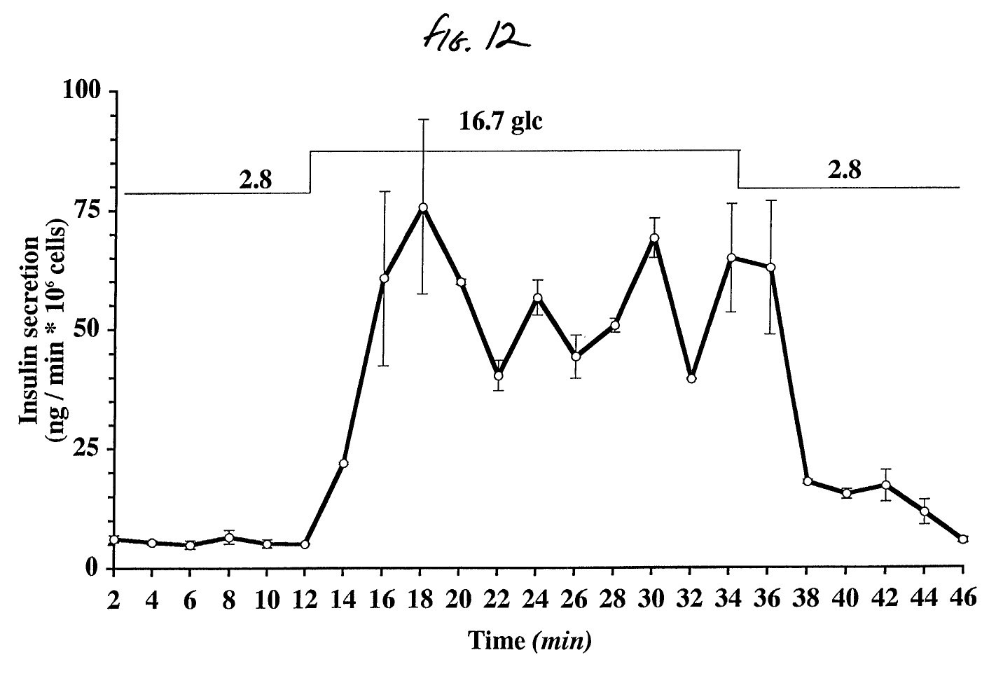

Clusters of β-cells were prepared and embedded at different cell densities in 1% agarose capsules. Under these conditions, the wild-type β-cells were barely able to survive densities above 1×10⁶ cells/ml, as determined by histological analysis (H&E) and measurement of glucose stimulated insulin secretion after 23 days of culture (FIG. 10). In contrast, the bcl-2 expressing cells were able to fully survive at densities up to 1×10⁷ cells/ml in agar and good cell survival was still detected at densities up to 2×10⁷. There was a good correlation between the ability of those cells to secrete insulin in response to an increase in glucose concentration and the ability to survive at increasing densities.

The bcl-2 cells could secrete up to 8.45U±2 (n=11) of insulin/24 hours at a density of 5×10⁶ cells/ml, whereas control cells at this density secreted only 0.14U±0,03 (n=12). Thus, overexpression of bcl-2 resulted in cells that can secrete up to 60 fold more insulin per 24 hours at a density of 5×10⁶ cells/ml. Additionally, overexpression of bcl-2 did not disturb the glucose sensitivity of the βTc-tet cells. Parallel perfusion of cells at equal number of passage showed similar perfusion pattern with a well preserved first and second phase of secretion.

### EXAMPLE 6

Bcl-2 Transformed Cells Implanted Into Chemically-Induced Diabetic Mice

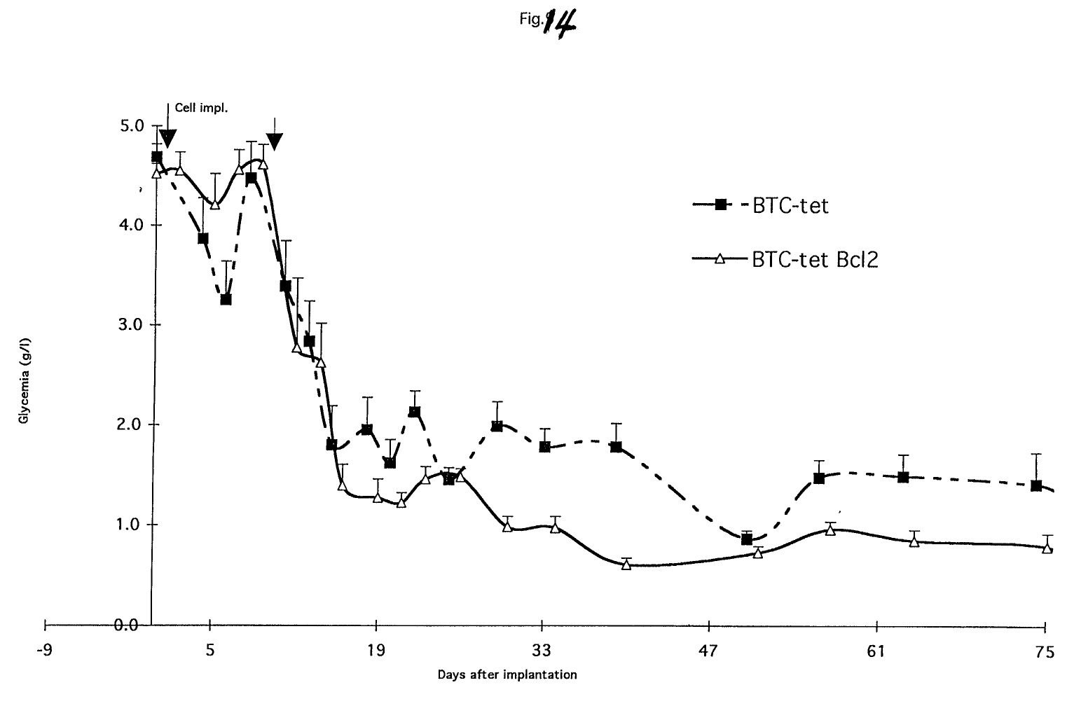

To generate diabetic mice, C3H male mice were injected i.p. with streptozotocin (Sigma) as described by Efrat et al., Proc. Natl. Acad. Sci. USA 92: 3576-3580 (1995), incorporated herein by reference. Wild-type β-cell clusters and bcl-2 β-cell clusters corresponding to 2×10⁶ cells were implanted under the kidney capsule using published standard techniques. Mice were monitored by-weekly for blood glucose with Glucometer strips. After approximately a two week period, the mice began to show β-cell activity. When glycemia was below 3 mg/ml, show release tetracycline pellets were implanted subcutaneously To control the mass of implanted β-cells. Thereafter, both groups of mice showed a stabilized normoglycemia over a three month period. Mice implanted with the bcl-2 transformed cells demonstrated a slightly lower post-prandial basal glucose level than the control group and average glucose level 75 days after transplantation was 1.4±0.3 g/l (n=8) for the untransformed control group, and 0.8±0.12 g/l (n=20) for the bcl-2 transformed group, as depicted in FIG. 14.

EQUIVALENTS

From the foregoing detailed description of the specific embodiments of the invention, it should be apparent that a novel genetically engineered cell, a cell implantation device, and method has been described. Although particular embodiments have been disclosed herein in detail, this has been done by way of example for purposes of illustration only, and is not intended to be limiting with respect to the scope of the appended claims which follow. In particular, it is contemplated by the inventor that various substitutions, alterations, and modifications may be made to the invention without departing from the spirit and scope of the invention as defined by the claims. For instance, the choice of the particular cell, anti-apoptosis gene, or the particular promoter is believed to be a matter of routine for a person of ordinary skill in the art with knowledge of the embodiments described herein.

What is claimed is:

1. A cell stably transformed to produce an anti-apoptosis polypeptide in response to hypoxic conditions, the cell exhibiting increased resistance to apoptosis.

2. The cell of claim 1 wherein the anti-apoptosis polypeptide is selected from the group consisting of bfl-1, bclX_{L}, Bax, Mcl-1, A1, ced-9, LMW5-HL, and BHRF-1.

3. The cell of claim 1 wherein the cell is a βTc-tet cell.

4. The cell of claim 1 wherein the cell is a muscle fibroblast.

5. A cell transformed with DNA encoding an anti-apoptosis gene, wherein the gene is operably linked to a promoter that is activated under hypoxic conditions.

6. The cell of claim 5 wherein the gene is selected from the group consisting of bfl-1, bclX_{L}, Bax, Mcl-1, A1, ced-9, LMW5-HL, and BHRF-1.

7. The cell of claim 5 wherein the promoter is selected from the group consisting of the promoter for PGK, the promoter for hsp 70, and the promoter for fos.

8. The cell of claim 5 wherein the cell is a βTc-tet cell.

9. The cell of claim 5 wherein the cell is a muscle fibroblast.

10. A cell transformed with DNA encoding a bcl-2 gene, wherein the bcl-2 gene is operably linked to a PGK promoter.

11. The cell of claim 10 wherein the bcl-2 gene is operably linked to the expression enhancing sequence of the WHV.

12. The cell of claim 10 or 11 wherein the cell is a βTc-tet cell.

13. The cell of claim 10 or 11 wherein the cell is a muscle fibroblast.

14. A method of treating IDDM comprising implanting at an implantation site in a patient a therapeutically effective number of cells of any of claims 3, 8, or 12.

15. The method of claim 14 wherein the cells are encapsulated in a semi-permeable membrane to form a bioartificial organ.

16. The method of claim 15 wherein the semipermeable membrane is immunoisolatory.

17. A bioartificial organ comprising:
a) a plurality of cells stably transformed to produce an anti-apoptosis polypeptide in response to hypoxic conditions, the cells exhibiting increased resistance to apoptosis; and
b) a semipermeable immunoisolatory membrane surrounding the plurality of cells.

18. The bioartificial organ of claim 17 wherein the anti-apoptosis polypeptide is selected from the group consisting of bfl-1, bclX_{L}, Bax, Mcl-1, A1, ced-9, LMW5-HL, and BHRF-1.

19. The bioartificial organ of claim 17 wherein the cells are selected from the group consisting of βTc-tet cells and muscle fibroblasts.
